(19) United States
(12) Patent Application Publication
(10) Pub. No.: US 2008/0228272 A1
(43) Pub. Date: Sep. 18, 2008

(54) DYNAMICALLY ADJUSTABLE SUTURE AND CHORDAE TENDINAE

(75) Inventors: Shahram Moaddeb, Irvine, CA (US); Samuel Shaolian, Newport Beach, CA (US)

Correspondence Address: MCDERMOTT WILL & EMERY LLP, 18191 VON KARMAN AVE., SUITE 500, IRVINE, CA, 92612-7108, US

(73) Assignee: Micardia Corporation, Irvine, CA (US)

(21) Appl. No.: 11/950,407

(22) Filed: Dec. 4, 2007

Related U.S. Application Data
(60) Provisional application No. 60/872,839, filed on Dec. 4, 2006.

(51) Int. Cl. A61F 2/08

(52) U.S. Cl. 623/13.13

(57) ABSTRACT

Embodiments of a dynamically adjustable artificial chordae tendinae implant are described. In some embodiments the implant includes a body portion, including an adjustable portion. In some embodiments, the implant includes a plurality of adjustable portions. In some embodiments the adjustable element can include a shape memory material. The adjustable portion can be configured to transform from a first conformation to a second conformation in response to an activation energy. In some embodiments, the activation energy can be one of electromagnetic energy, acoustic energy, light energy, thermal energy, electrical energy, mechanical energy, or a combination of energies. The implant couples a heart valve leaflet to a papillary muscle. Activation of the shape memory material regulates tension between the muscle and valve leaflet improving coaptation of heart valve leaflets, and reducing or eliminating regurgitation.

### RELATED APPLICATIONS

This application claims the priority benefit of U.S. Provisional Patent Application No. 60/872,839, filed Dec. 4, 2006, entitled “Dynamically Adjustable Suture and Chordae Tendinae Filament,” the contents of which are incorporated by reference herein in their entirety.

### FIELD OF THE INVENTION

Embodiments of the invention relate to devices and methods for use in the repair of cardiac valves, in particular an artificial chordae tendinae and methods for implanting the same.

### BACKGROUND OF THE INVENTION

The human heart has four valves that control the direction of blood flow in the circulatory system. The aortic and mitral valves are part of the “left” heart and control the flow of oxygen-rich blood from the lungs to the peripheral circulation, while the pulmonary and tricuspid valves are part of the “right” heart and control the flow of oxygen-depleted blood, returning from the body, to the lungs. The aortic and pulmonary valves lie between a pumping chamber (ventricle) and major artery, preventing blood from leaking back into the ventricle after being ejected into the circulation. The mitral and tricuspid valves lie between a receiving chamber (atrium) and a ventricle preventing blood from flowing back into the atrium during ventricular contraction.

Various disease processes can impair the proper functioning of one or more of these valves. These include degenerative processes (e.g., Barlow's Disease, fibroelastic deficiency), inflammatory processes (e.g., rheumatic heart disease), and infectious processes (e.g., endocarditis). In addition, damage to the ventricle from prior heart attacks, or other heart diseases (e.g., cardiomyopathy), can distort valve geometry leading to diminished functionality.

Heart valves can malfunction in one of two ways. Valve stenosis describes the situation where the valve does not open completely, resulting in an obstruction to blood flow. Valve regurgitation describes the situation where the valve does not close completely, resulting in leakage back into a heart chamber against the normal direction of flow (e.g., leakage from a ventricle back to an atrium, or from the circulation back to a ventricle). Both of these conditions increase the workload on the heart and, if left untreated, can lead to conditions including congestive heart failure, permanent heart damage, and ultimately death. Dysfunction of the left-sided valves—the aortic and mitral valves—is typically more serious since the left ventricle is the primary pumping chamber of the heart.

Treatment options can include valve repair, preserving the patient's natural valve, or replacement with a mechanical, or biologically-derived, substitute valve. Since there are well known disadvantages associated with the use of valve prostheses, including increased clotting risk, and limited durability of the replacement valve, repair is usually preferable, when possible, to replacement. In many cases, however, valves are diseased or damaged beyond repair such that the only viable option remaining is replacement. In addition, valve repair is usually more technically demanding than replacement. Thus, the number of surgeons capable of performing complex valve repairs is limited. As a result, the appropriate treatment depends on the specific valve involved, the specific disease/dysfunction, the degree of disease and/or damage, and the experience of the surgeon.

The aortic valve is more prone to stenosis, which typically results from buildup of calcified material on the valve leaflets, and usually requires aortic valve replacement. Regurgitant aortic valves can sometimes be repaired but generally replacement is indicated. The pulmonary valve has a structure and function similar to that of the aortic valve. Dysfunction of the pulmonary valve, however, is much less common and is nearly always associated with complex congenital heart defects. Pulmonary valve replacement is occasionally performed in adults with longstanding congenital heart disease.

Mitral valve regurgitation is more common than mitral stenosis. Although mitral stenosis, which usually results from inflammation and fusion of the valve leaflets, can often be repaired by peeling the leaflets apart from each other (commissurotomy), as with aortic stenosis, the valve is often heavily damaged and can require replacement. Mitral regurgitation, however, can nearly always be repaired.

The normal mitral valve 2, an example of which is illustrated in FIGS. 1A and 1B, can be divided into three parts, an annulus 4, a pair of leaflets 6, 8 and a sub-valvular apparatus. The annulus 4 is a dense ring of fibrous tissue which lies at the juncture between the left atrium and the left ventricle. The annulus 4 is normally elliptical, or “kidney-shaped,” with a vertical (anteroposterior) diameter approximately three-fourths of the transverse diameter. The larger elliptical anterior leaflet 6 and the smaller, crescent-shaped posterior leaflet 8 attach to the annulus 4. Approximately three-fifths of the circumference of annulus 4 is attached to the posterior leaflet 8 and two-fifths of the annular circumference is attached to the anterior leaflet 6. The edge of each leaflet not attached to the annulus 4 is known as the free margin 10.

When the valve is closed, the free margins of the two leaflets come together within the valve orifice forming an arc known as the line of coaptation 12. The points on the annulus where the anterior and posterior leaflets meet, are known as commissures 14. The posterior leaflet 8 is usually separated into three distinct scallops by small clefts. The posterior scallops are referred to (from left to right) as P1 (anterior scallop), P2 (middle scallop) and P3 (posterior scallop). The corresponding segments of the anterior leaflet directly opposite P1, P2 and P3 are referred to as A1 (anterior segment), A2 (middle segment) and A3 (posterior segment).

The sub-valvular apparatus consists of two thumb-like muscular projections from the inner wall of the left ventricle (not shown) known as papillary muscles 16 and numerous chordae tendinae 18, thin fibrous bundles that emanate from the tips of the papillary muscles 16 and attach to the free margin 10 or undersurface of the valve leaflets in a parachute-like configuration. The chordae 18 are classified according to their site of attachment between the free margin 10 and the base of the leaflets. Marginal, or primary, chordae are attached at the free margin 10 of the leaflets and function to limit leaflet prolapse. Intermediate, or secondary, chordae are attached or attached to the underside of the leaflets at points between the free margin 10 and the base of the leaflets. Basal, or tertiary, chordae are attached to the base of the leaflets.

Normally, the mitral valve opens when the left ventricle relaxes (diastole) allowing blood from the left atrium to fill the left ventricle. When the left ventricle contracts (systole), the increase in pressure within the ventricle causes the valve to close, preventing blood leakage back into the left atrium, and ensuring that substantially all of the blood leaving the left ventricle (the stroke volume) is ejected through the aortic valve into the aorta and to the peripheral circulation of the body. Proper function of the valve is dependent on a complex interplay between the annulus, leaflets and subvalvular apparatus.

Lesions in any of these components can lead to valve dysfunction, resulting in a backflow of blood from the left ventricle to the left atrium during systole, a condition known as mitral regurgitation. Since a portion of cardiac output is wasted when blood flows back into the left atrium, the heart must work harder in order to the volume of blood needed to maintain proper perfusion of tissues in the body. Over time, this increased workload leads to myocardial remodeling in the form of left ventricular dilation, or hypertrophy. It also leads to increased pressures in the left atrium, resulting in the back up of blood in the venous circulation, and fluid in the tissues of the body, a condition known as congestive heart failure.

Mitral valve dysfunction leading to mitral regurgitation can be classified into three types based on the motion of the leaflets (known as “Carpentier's Functional Classification”). Type I dysfunction generally does not affect normal leaflet motion. Mitral regurgitation in these patients can be due to perforation of the leaflet (usually from infection), or much more commonly, result from distortion or dilation of the annulus. Annular dilation/distortion causes separation of the free margins of the two leaflets, producing a gap. This gap prevents the leaflets from fully coapting, in turn allowing blood to leak back into the left atrium during systolic contraction. Type II dysfunction results from leaflet prolapse. This occurs when a portion of the free margin of one, or both, leaflets is not properly supported by the subvalvular apparatus. During systolic contraction, the free margins of the involved portions of the leaflets prolapse above the plane of the annulus and into the left atrium. This prevents leaflet coaptation and again allows blood to regurgitate into the left atrium between the leaflets. The most common lesions resulting in Type II dysfunction include chordal or papillary muscle elongation, or rupture, due to degenerative changes (such as myxomatous pathology or Barlow's Disease and fibroelastic deficiency), or prior myocardial infarction. Type III dysfunction results from restricted leaflet motion. Here, the free margins of portions of one or both leaflets are pulled below the plane of the annulus into the left ventricle. Leaflet motion that is restricted during both systole and diastole is termed a Type III A dysfunction. The restricted leaflet motion can be related to valvular or subvalvular pathology including leaflet thickening or retraction, chordal thickening, shortening or fusion and commissural fission, any or all of which can be associated with some degree of stenosis or fibrosis. Leaflet motion which is restricted during systole only is termed a Type III B dysfunction. Specifically, the leaflets are prevented from rising up to the plane of the annulus and coapting during systolic contraction. This type of dysfunction most commonly occurs when abnormal ventricular geometry or function, usually resulting from prior myocardial infarction (“ischemia”) or severe ventricular dilatation and dysfunction (“cardiomyopathy”), leads to papillary muscle displacement. The otherwise normal leaflets are pulled down into the ventricle and away from each other, preventing proper coaptation.

The anatomy and function of the tricuspid valve is similar to that of the mitral valve. It also has an annulus, chordae and papillary muscles but with three leaflets (anterior, posterior and septal). The mechanical loads imposed on the tricuspid valve are significantly less than the mitral valve since the pressures in the right heart are normally only about 20% of those of the left heart.

Tricuspid stenosis is very rare in adults and usually results from very advanced rheumatic heart disease. Tricuspid regurgitation is much more common and can result from the same types of dysfunction (I, II, IIIA and IIIB) as the mitral valve. The vast majority of patients suffering from tricuspid regurgitation, however, have Type I dysfunction with annular dilation preventing normal leaflet coaptation. This is usually secondary to left heart disease (valvular or ventricular) which can, over time, lead to increased upstream pressures, for example, in the pulmonary arteries, right ventricle and right atrium. The increased pressures in the right heart can lead to dilation of the chambers and concomitant tricuspid annular dilation.

A common cause of insufficiency of the mitral valves is due to Type II dysfunction (leaflet prolapse). Repair of this dysfunction usually requires some type of leaflet resection and reconstruction along with, on occasion, additional leaflet and chordal procedures. The most common type of valve repair for Type II valve dysfunction is a quadrangular resection of the middle (P2) segment of the posterior leaflet. Resection of the P2 segment involves making perpendicular incisions from the free edge of the posterior leaflet toward the annulus, and then excising a quadrangular portion of the leaflet. Plication sutures are placed along the posterior annulus in the resected area, and direct sutures are applied to the leaflet remnants, to restore valve continuity.

When excessive posterior leaflet tissue is present, such as in patients suffering from Barlow's disease, an ancillary procedure referred to as a sliding valvuloplasty is also performed. In this procedure, the P1 and P3 segments of the posterior leaflet are detached from the annulus, and compression sutures are placed in the posterior segment of the annulus. The gap between the two segments is then closed with interrupted sutures. As such, the height of the posterior leaflet is reduced to avoid postoperative systolic anterior motion (SAM). Sliding valvuloplasty is also indicated if a large quadrangle segment of the posterior leaflet is excised.

While many surgeons are comfortable repairing straightforward cases of P2 prolapse as described above, more complex Type II cases, including those with anterior leaflet involvement or prolapse at or near the commissures, usually require additional procedures that can be outside the expertise of the average surgeon. These can include chordal transfer, chordal transposition, placement of artificial chords, triangular resection of the anterior leaflet, sliding plasty or shortening of the papillary muscle and sliding plasty of the paracommissural area. As a result, most surgeons, outside of specialized centers, rarely tackle these complex repairs and so these patients usually receive a valve replacement.

In the early 1990s, the concept of edge-to-edge repair was popularized, a procedure first described 50 years ago (Nichols, H. T. (1957). Mitral insufficiency: treatment by polar cross-fusion of the mitral annulus fibrosus. J. Thorac. Surg. 33: 102-122). This repair technique consists of suturing together the edges of the leaflets at the site of regurgitation. The procedure can be use to effect repairs both at the paracommissural area (at the A1 and P1 segments of the leaflets), and at the middle of the valve (at the A2 and P2 segments, a procedure referred to as a “double orifice repair.”

Initial studies revealed a high rate of failure of the edge-to-edge repair, particularly in patients with mitral regurgitation resulting from rheumatic fever. Thus, it was generally recommended that a concomitant annuloplasty be performed in every patient. More recently, the double orifice edge-to-edge technique has been applied to patients with Barlow's disease (typically involving prolapse of multiple segments) and bi-leaflet prolapse with satisfactory results.

Conventional procedures for replacing or repairing cardiac valves require the use of the heart-lung machine (cardiopulmonary bypass) and stopping the heart by clamping the ascending aorta (“cross-clamping”) and perfusing with a high-potassium solution (cardioplegic arrest). Although most patients tolerate limited periods of cardiopulmonary bypass and cardiac arrest well, these procedures are known to adversely affect all organ systems. The most common complications of cardiopulmonary bypass and cardiac arrest are stroke, myocardial “stunning” or damage, respiratory failure, kidney failure, bleeding and generalized inflammation. If severe, these complications can lead to permanent disability or death. The risk of these complications is directly related to the amount of time the patient is on the heart-lung machine (“pump time”) and the amount of time the heart is stopped (“cross-clamp time”). Although the safe windows for pump time and cross clamp time depend on individual patient characteristics (age, cardiac reserve, co-morbid conditions, etc.), pump times over 4 hours and clamp times over 3 hours are generally of concern in all patients.

Within recent years, there has been a movement to perform many cardiac surgical procedures using “minimally invasive” techniques. These are characterized by the use of smaller incisions and innovative cardiopulmonary bypass protocols. The purported benefits of these approaches include less pain, less trauma and more rapid recovery. This has included “off-pump coronary artery bypass” (OPCAB) surgery which is performed on a beating heart without the use of cardiopulmonary bypass and “minimally invasive direct coronary artery bypass” (MIDCAB) which is performed through a small thoracotomy incision. A variety of minimally invasive valve repair procedures have been developed whereby the procedure is performed through a small incision with or without videoscopic assistance and, more recently, robotic assistance.

### SUMMARY OF THE INVENTION

In spite of advances in cardiovascular repair techniques, there remain significant limitations to the usefulness of currently available methods and devices for use in repairing heart defects arising from injury or disease. For example, it has been found that the edge-to-edge repair, particularly the double orifice technique, results in a significant decrease in mitral valve area, which can lead to mitral stenosis. Even without physiologic mitral stenosis, the decrease in orifice area increases flow velocities and turbulence, which can lead to fibrosis and calcification of functioning valve segments. Turbulence can also lead to an increased risk of blood clot formation. This will likely impact the long-term durability of this repair.

Another factor, which can impact the long-term durability of the edge-to-edge technique, is the increased stress on the subvalvular apparatus of all segments. For example, in a patient with isolated A2 prolapse, suturing A2 to P2 increases the stress on the latter segment. As a result, current clinical data does not support the routine use of the edge-to-edge technique for the treatment of Type II mitral regurgitation.

As described above, in conventional procedures, additional complications can result from extended use of cardiac bypass for durations in excess of 3 to 4 hours. Complex valve repairs can push the time limits even in the most experienced hands. As a result, a less experienced surgeon is often reluctant to spend 3 hours trying to repair a valve since, if the repair is unsuccessful, they will have to spend up to an additional hour replacing the valve, increasing the risk of complications due to the length of time spent on a heart-lung machine. Time becomes a significant factor in choosing valve repair over replacement, and thus, devices and techniques that simplify and expedite valve repair will be desirable.

In addition, the use of minimally invasive procedures has been limited to a handful of surgeons at specialized centers in a very selected group of patients. Even in their hands, the most complex valve repairs cannot be performed since dexterity is limited and thus the procedure moves slowly. As a result, devices and techniques that simplify valve repair have the potential to greatly increase the use of minimally invasive techniques which would significantly benefit patients.

Currently, heart valve repair includes several different techniques, among which are annuloplasty and chordae tendinae replacement. Chordae tendinae play an important role in correct valve coaptation by connecting the heart valve leaflets to the papillary muscles. The papillary muscle exert tension on the chordae to prevent inversion of the valve leaflets. In mitral valve regurgitation associated with ischemia, the chordae tendinae cannot function properly. In these situations, artificial sutures such as ePTFE (Gore-Tex®) have been used as replacements for damaged natural chordae tendinae. However, there are limitations to presently available artificial chordae tendinae. These include the inability to dynamically adjust their size and orientation, and a lack of mechanical strength to sufficiently lift and modify the left ventricle. As a result, changes in the size and shape of the left ventricle as a result of ischemia continue.

Thus, there is a need for artificial chordae tendinae that can be dynamically adjusted, and which have sufficient mechanical strength. Embodiments as described herein address the above-described deficiencies of current therapies, particularly, the malfunctioning of chordae tendinae, by providing permanent implants that can be dynamically adjusted postoperatively via internal or external means. These dynamically adjustable artificial chordae tendinae are effective to improve coaptation of heart valve leaflets, and reduce or event prevent regurgitation.

Accordingly, in some embodiments there is provided a dynamically adjustable artificial chordae tendinae implant, for use in treating a heart valve in a patient, comprising: a body portion, having first and second ends, and comprising: a first attachment portion that couples the body portion to a leaflet of a valve in a heart; a second attachment portion that couples the body portion to a papillary muscle in the heart; and an adjustable portion, comprising a shape memory material; wherein, in response to an activation energy, the adjustable portion transforms from a first conformation to a second conformation; wherein in the first conformation the ends of body portion are separated by a first length; and wherein in the second conformation the ends of the body portion are separated by a second length.

In some embodiments, transformation from the first conformation to the second conformation results in improved coaptation of the leaflet of the valve with at least one other leaflet of the same valve.

In some embodiments, the shape memory material comprises at least one of a shape memory alloy, a ferromagnetic shape memory alloy, a shape memory polymer, and a combination thereof.

In some embodiments, the adjustable portion is configured to transform from the first conformation to the second conformation at a first activation temperature. In some embodiments, the adjustable portion is configured to transform to a third conformation at a second activation temperature. In some embodiments, in the third conformation, the ends of the body portion are separated by a third length.

In some embodiments, the first length is greater than the second length, for at least a portion of a cardiac cycle. In some embodiments, the first length is less than the second length, for at least a portion of a cardiac cycle. In some embodiments, the third length is greater than the second length, for at least a portion of a cardiac cycle. In some embodiments, the third length is greater than the first length, for at least a portion of a cardiac cycle. In some embodiments, the third length is less than the first length, for at least a portion of a cardiac cycle.

In some embodiments, transformation from the first conformation to the second conformation occurs incrementally. In some embodiments, transformation to the third conformation occurs incrementally.

In some embodiments, at least one of the first attachment portion and the second attachment portion comprises a suture. In some embodiments, the suture comprises at least one of catgut, silk, linen, stainless steel wire, polyglycolic acid, polyglactin, polydioxanone, polyglyconate, polyamide, polyester, polypropylene, ePTFE, and a combination thereof.

In some embodiments, the implant further comprises a cover over at least a portion of the implant. In some embodiments, the cover comprises at least one of a biodegradable material, a biocompatible material, a thermal insulator, an electrical insulator, and a combination thereof. In some embodiments, the cover comprises a gap configured to expose a portion of the implant. In some embodiments, the cover can be configured to be suturable to at least one of the valve leaflet and the papillary muscle.

In some embodiments, the implant further comprises at least one medicament in or on at least a portion of the implant, the medicament effective to promote healing, reduce inflammation, or reduce thrombosis, in the patient.

In some embodiments, the implant further comprises an energy absorbing material coupled to the adjustable portion. In some embodiments, the energy absorbing material is configured to provide thermal energy to the adjustable portion. In some embodiments, the energy absorbing material comprises at least one of a hydrogel, carbon, graphite, a ceramic material, a magnetic material, a microporous coating, a magnetic induction coil, an electrically conductive wire, nanospheres, and combinations thereof. In some embodiments, the energy absorbing material is configured to absorb at least one of electromagnetic energy, radiofrequency energy, acoustic energy, light energy, thermal energy, electrical energy, mechanical energy, and a combination thereof. In some embodiments, the acoustic energy comprises high intensity focused ultrasound energy.

In some embodiments, the implant comprises a plurality of adjustable portions, each of the plurality of adjustable portions comprising a shape memory material; wherein, in response to an activation energy, each of the plurality of adjustable portions transforms from an initial conformation to a transformed conformation. In some embodiments, each of the plurality of adjustable portions transforms independently from the initial conformation to the transformed conformation. In some embodiments, the plurality of adjustable portions are arranged in segments along at least a portion of the body portion. In some embodiments, each adjustable portion segment is separated from an adjacent adjustable portion segment by a non-adjustable portion. In some embodiments, the non-adjustable portion comprises an insulator.

In some embodiments, the implant further comprises at least one sensor configured to output data to a receiver, the data indicative of at least one of a temperature of the implant and a temperature of a body tissue in thermal communication with the implant.

In some embodiments, there is provided a dynamically adjustable artificial chordae tendinae implant system, comprising: an implant, comprising: a body portion, having first and second ends, and comprising: a first attachment portion that couples the body portion to a leaflet of a valve in a heart; a second attachment portion that couples the body portion to a papillary muscle in the heart; and an adjustable portion, comprising a shape memory material; wherein, in response to an activation energy, the adjustable portion transforms from a first conformation to a second conformation; wherein in the first conformation the ends of body portion are separated by a first length; and wherein in the second conformation the ends of the body portion are separated by a second length; and a energy delivery system configured to deliver the activation energy to the implant.

In some embodiments, the energy delivery system delivers at least one of electromagnetic energy, radiofrequency energy, acoustic energy, light energy, thermal energy, electrical energy, and mechanical energy to the implant.

In some embodiments, the system further comprises at least one sensor configured to output data indicative of at least one of a temperature of the implant and a temperature in a tissue in thermal communication with the implant.

In some embodiments, the energy delivery system is configured to terminate or reduce energy delivery upon receipt of data from the at least one sensor indicative of at least one of attaining a target temperature in the implant and exceeding a threshold temperature in the tissue.

In some embodiments, the system further comprises a display module for displaying the data.

In some embodiments, there is provided a dynamically adjustable artificial chordae tendinae implant system, comprising: coupling means for coupling a heart valve leaflet to a papillary muscle in a patient, the coupling means having first and second ends separated by a first length; adjusting means for changing the length of the coupling means; wherein the adjusting means comprises a shape memory material that transforms from a first conformation to a second conformation in response to an activation energy; and wherein, when the shape memory material transforms from the first conformation to the second conformation, the implant improves coaptation of the heart valve leaflet with at least one other heart valve leaflet.

In some embodiments, the system is configured such that when the shape memory material is in the second conformation, the ends of the coupling means are separated by a second length.

In some embodiments, the system further comprises energy delivery means for delivering the activation energy to the implant. In some embodiments, the activation energy is at least one of electromagnetic energy, radiofrequency energy, acoustic energy, light energy, thermal energy, electrical energy, mechanical energy, and a combination thereof. In some embodiments, the energy delivery means is configured to deliver the activation energy of the implant from a location outside the patient's body.

In some embodiments, the system further comprises sensing means for outputting data indicative of at least one of a temperature of the implant and a temperature of a tissue in thermal communication with the implant.

In some embodiments, the system further comprises display means for displaying the at least one of the temperature of the implant and the temperature of the tissue.

In some embodiments, the system further comprises control means for terminating or reducing delivery of the energy to the implant in response to output data from the sensing means indicative of at least one of achieving a target temperature in the implant and exceeding a threshold temperature in the tissue.

In some embodiments of the system, the shape memory material comprises at least one of a shape memory alloy, a ferromagnetic shape memory alloy, a shape memory polymer, and a combination thereof. In some embodiments, the shape memory material is configured to transform from the first conformation to the second conformation at a first activation temperature. In some embodiments, the shape memory material is configured to transform to a third conformation at a second activation temperature. In some embodiments, when the shape memory material is in the third conformation, the ends of the coupling means are separated by a third length.

In some embodiments, the system further comprises attachment means for attaching the implant to at least one of the heart valve leaflet and the papillary muscle. In some embodiments, the attachment means comprises a suture.

In some embodiments, the system further comprises a covering means for covering at least a portion of the coupling means. In some embodiments, the covering means comprises at least one a biodegradable material, a biocompatible material, and an insulator.

In some embodiments, the implant comprises a plurality of adjusting means, each of the plurality of adjusting means comprising a shape memory material; wherein, in response to an activation energy, each of the plurality of adjusting means transforms from an initial conformation to a transformed conformation.

In some embodiments, each of the plurality of adjusting means transforms independently from the initial conformation to the transformed conformation. In some embodiments, the plurality of adjusting means are arranged in segments along at least a portion of the coupling means. In some embodiments, the system further comprises separating means for separating each of the plurality of adjusting means. In some embodiments, the separating means comprises a thermal insulator.

In some embodiments, there is provided a method, for implanting an artificial chordae tendinae in a patient, comprising: providing a dynamically adjustable artificial chordae tendinae implant, comprising: a body portion, having first and second ends, and comprising: a first attachment portion that couples the body portion to a leaflet of a valve in a heart; a second attachment portion that couples the body portion to a papillary muscle in the heart; and an adjustable portion, comprising a shape memory material; wherein, in response to an activation energy, the adjustable portion transforms from a first conformation to a second conformation; wherein in the first conformation the ends of body portion are separated by a first length; and wherein in the second conformation the ends of the body portion are separated by a second length; securing the first attachment portion to the heart valve leaflet; securing the second attachment portion to the papillary muscle; delivering the activation energy to the adjustable portion of the implant, resulting in a transformation from the first conformation to the second conformation; wherein transformation from the first conformation to the second conformation results in improved coaptation of the leaflet of the cardiac valve with at least one other leaflet of the same cardiac valve.

In some embodiments of the method, the activation energy comprises at least one of electromagnetic energy, radiofrequency energy, acoustic energy, light energy, thermal energy, electrical energy, mechanical energy, and a combination thereof. In some embodiments of the method, the activation energy is delivered from outside the patient's body.

In some embodiments, the method further comprises imaging at least one of the implant and a parameter indicative of a heart valve function. In some embodiments of the method, the adjusting is temporally coordinated with the imaging. In some embodiments of the method, the adjusting is performed relative to the occurrence of a physiological parameter. In some embodiments of the method, the physiological parameter is at least one of a cardiac cycle and the patient's breathing. In some embodiments of the method, the adjusting is performed while the patient holds his breath. In some embodiments of the method, the adjusting is performed during a QT interval of the cardiac cycle.

In some embodiments, the method further comprises providing at least one sensor configured to output data corresponding to at least one of a temperature of the implant and a temperature of a tissue in thermal communication with the implant. In some embodiments, the method further comprises terminating or reducing the delivery of activation energy to the implant in response to output data from the at least one sensor indicative of at least one of achieving a target temperature in the implant and exceeding a threshold temperature in the tissue.

### BRIEF DESCRIPTION OF THE DRAWINGS

FIG. 1A illustrates a perspective view of a normal mitral valve having proper coaptation of the anterior and posterior leaflets.

FIG. 1B illustrates a cross-sectional view of the heart, further illustrating the mitral valve.

FIG. 2A illustrates a side view of an artificial chordae tendinae.

FIG. 2B illustrates an end view of a cross-section of an artificial chordae tendinae.

FIGS. 2C-D illustrate embodiments of a dynamically adjustable artificial chordae tendinae after activation.

FIGS. 3A-B illustrate embodiments of a dynamically adjustable artificial chordae tendinae comprising a suturable material.

FIGS. 3C-E illustrate embodiments of dynamically adjustable artificial chordae tendinae comprising a suturable material, after activation.

FIG. 3F illustrates an embodiment of a dynamically adjustable artificial chordae tendinae comprising suture material and shape memory material with suturable disposed at an end.

FIGS. 3G-H illustrate an embodiment of a dynamically adjustable chordae tendinae with a plurality of adjustable portions.

FIG. 4A illustrates a left ventricle where dynamically adjustable chordae tendinae are attached on one end to mitral valve leaflet, and on an opposite end to papillary muscle.

FIG. 4B illustrates an example of a dynamically adjustable chordae tendinae implant like that of FIG. 4A after activation of the shape memory material.

FIG. 4C illustrates an embodiment dynamically adjustable artificial chordae tendinae attached at one end to a mitral valve leaflet, and to papillary muscle at the opposite end.

FIG. 5A illustrates an embodiment of percutaneous placement of adjustable sutures around a mitral valve annulus configured for postoperative adjustment.

FIG. 5B illustrates an example of a percutaneously placed adjustable sutures placed around a mitral valve and pulling on the annulus after postoperative adjustment.

FIGS. 6A-D illustrate activation of a dynamically adjustable artificial chordae tendinae implant via a device adapted to focus energy onto the implant.

FIG. 7 illustrates an embodiment of a dynamically adjustable artificial chordae tendinae with suture or suture-like attachment structures.

### DETAILED DESCRIPTION OF THE INVENTION

In the present disclosure, embodiments of artificial dynamically adjustable chordae tendinae take advantage of the properties of shape memory materials in order to provide an improved implant for use in the repair of cardiac valve defects. In particular, embodiments of the implant allow for precise configuring of the artificial chordae to provide optimal correction of a valvular defect.

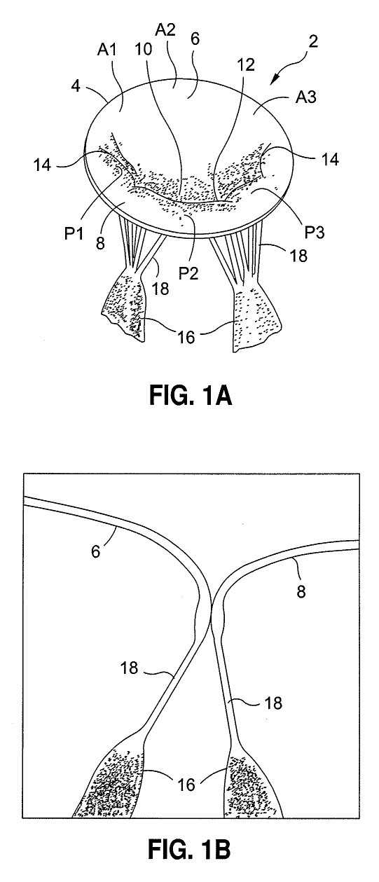

A normal mitral valve 2 is illustrated in FIGS. 1A and 1B, and can be divided into three parts, an annulus 4, a pair of leaflets 6, 8 and a sub-valvular apparatus.

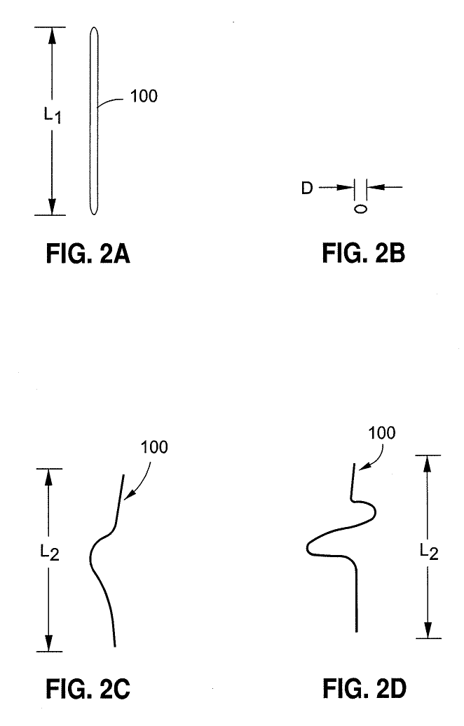

FIGS. 2A and 2B illustrate embodiments of dynamically adjustable artificial chordae tendinae 100 prior to activation of the shape memory component portions. Dynamically adjustable artificial chordae tendinae 100 can be any shape memory material, alloy, or polymer described above, and can be configured either as a monofilament or multifilament structure, or a collection of monofilament or multifilament structures. As illustrated in FIG. 2A, before activation, an artificial chordae tendinae 100 will have a length L₁. FIG. 2B illustrates a cross-section of dynamically adjustable artificial chordae tendinae 100, and indicating a diameter D, which in some embodiments will range from about 0.05 mm to about 0.4 mm.

FIGS. 2C and 2D illustrate embodiments of dynamically adjustable artificial chordae tendinae 100 after activation. FIGS. 2C and 2D illustrate that after activation of the implant the dynamically adjustable artificial chordae tendinae 100 will have a length L₂. In some embodiments, L₂ will be less than or equal to L₁, as illustrated in FIG. 2A. In some embodiments, L₂ will be greater than or equal to L₁. In some embodiments the length can be substantially unchanged by activation, but instead the elastic properties of the implant can be altered to make the implant either more or less compliant, as desired. Thus, changing the configuration of the implant by activation of the shape memory portion of the device can be used to either shorten or lengthen the artificial chordae tendinae, or to change the mechanical properties of the implant.

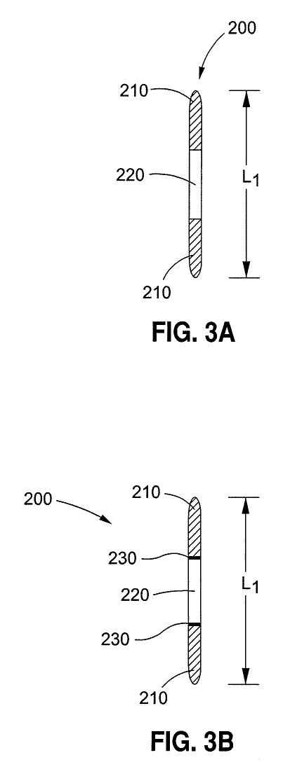

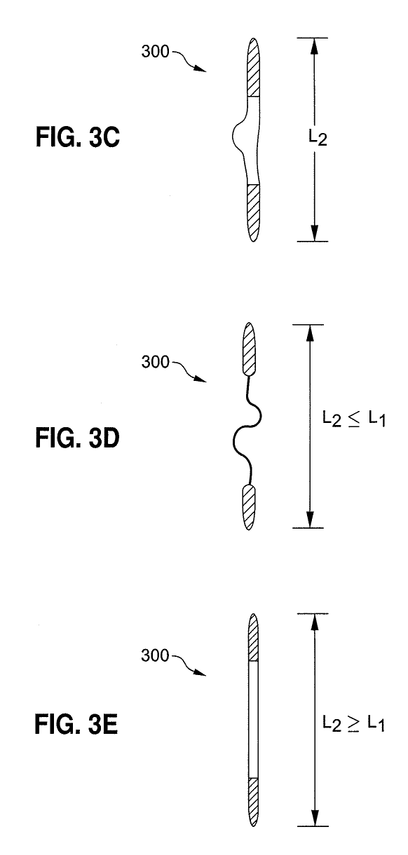

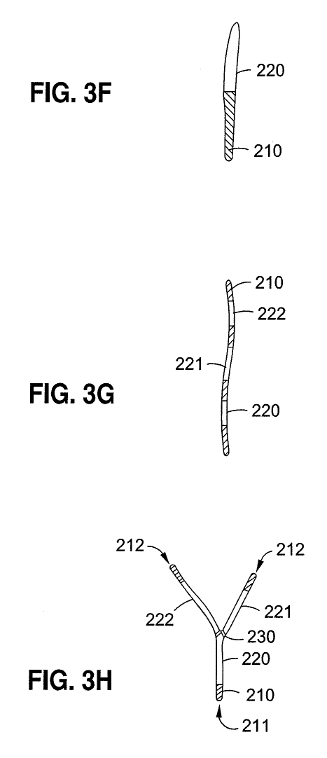

FIGS. 3A and 3B illustrate exemplary embodiments of composite artificial chordae tendinae 200. An artificial chordae tendinae 200 with length L₁ can comprise a suturable material 210, and a shape memory portion 220. Suitable suture materials that can be used include, but are not limited to catgut (plain or chromic), silk, linen, stainless steel wire, polyglycolic acid (Dexon), polyglactin (Vicryl®), polydioxanone (PDS), polyglyconate (Maxon™), polyamide (Nylon), polyester (Dacron), polypropylene (Prolene), or ePTFE (Gore-Tex®). Suitable suture material can also be any suitable natural or artificial fibers. Any of the these suture materials can optionally include coatings to enhance their performance characteristics. Additionally, these suture materials can be a monofilament or can be braided into a multifilament. Moreover, suture material can be selected for appropriate sizes, length, or can optionally include various pledget configurations.

The shape memory portion 220 can comprise any suitable shape memory material. In some embodiments, the shape memory portion 220 and suturable material 210 can be joined at an attachment 230, for example, as shown in FIG. 3B. The attachment 230 can comprise an adhesive material, or any other material or mechanism by which to couple the suturable material 210 to the shape memory material 230. Those of skill in the art will readily appreciate the numerous ways to fasten the various parts of the implant, and all such fasteners and fastening methods are considered to be including within the present disclosure.

In some embodiments, for example as shown in FIGS. 3A-F, the implant comprises a single adjustable portion, fashioned for example from a shape memory material. In some embodiments, one of which is shown in FIG. 3G, the implant can comprise a plurality of shape memory portions 220, 221, an 221, interspersed with suturable material 210, which, in some embodiments, comprises a cover, or sleeve. In some embodiments, for example, as shown in FIG. 3F, the suturable material can be disposed generally towards one end. In some embodiments, shape memory portions 220, 221, and 222 can be activated by different energy levels, different temperatures, and/or different energy types (e.g., magnetic, radiofrequency (RF), thermal, and/or acoustic energy). In some embodiments, these shape memory portions can be activated simultaneously or sequentially. In some embodiments, some or all of these shape memory portions can be activated during a surgical procedure, or postsurgery.

Having a plurality of shape memory regions permits independent activation of each region, such that the length, or mechanical properties, of an artificial chordae tendinae can be incrementally adjusted. In some embodiments, as in FIG. 3G, the plurality of shape memory regions are disposed in an segmental arrangement axially along at least a portion of the body of the implant. In some embodiments, the material forming the attachment 230 can be configured to insulate adjacent sections of shape memory material from each other, or from other parts of the body portion. In some embodiments, the body portion comprises a plurality of shape memory portions that are insulated from each other by insulating section 225. The shape memory portions can be configured to respond to the same energy, or they can be configured to respond to different energies, such that each shape memory portion if capable of being adjusted independently of other shape memory portion in the same implant. The precise configuration of adjustable portions can be varied without departing from the scope of the disclosure.

In some embodiments, the artificial chordae tendinae can be configured in a Y-shape, such that a muscle end 211 can be attached to a papillary muscle, and leaflet ends 212 can be attached to different valve leaflets, or to different portions of the same leaflet. In some embodiments there can be additional muscle ends 211, as well as more than 2 leaflet ends 212. Again, the precise configuration will be readily determinable by one of skill in the art when considering an optimal solution for a patient.

Conveniently, the implant can comprises a number of different shape memory materials, 220, 221, and 222, each configured to respond to different energies, or to change shape in response to different activation temperatures. Thus, each portion of the implant can be adjusted independently of the others. In use, this could allow for example, the tensioning of one leaflet by, or a different part of the same leaflet, by different amounts. For example, each cusp of a valve could be independently adjusted using a device like that shown in FIG. 3H.

Thus, in a multi-section shaped memory material corresponding connective-coupling sections, each individual shaped memory section can be adjusted using any suitable energy source (e.g., and without limitation, thermal energy, magnetic energy, electromagnetic energy, radio frequency energy, ultrasound energy, high energy focused ultrasound energy, computed tomography (CT) scanning, X-ray imaging, etc.). As indicated above, in some embodiments different sections of shape memory material can be configured to respond to different energy sources. Thus, for example, one portion of the implant can be configured to respond to ultrasound, while another responds to direct application of thermal energy, etc. All combinations and permutations of energy sources are thus contemplated as be useable with embodiments of artificial chordae tendinae implants as described herein.

FIGS. 3C-E illustrate embodiments of an adjustable artificial chordae tendinae 300 after activation of the shape memory material. In FIG. 3C the shape of the artificial chordae is shown to be altered, but the length, L₂ remains substantially equal to the initial length L₁. This can be useful where it is desired to activate a shape memory material in order to affect malleability of the material without substantially changing the length of the structure that the shape memory material comprises.

In FIG. 3D, the drawing depicts an example of an artificial chordae tendinae following activation of the shape memory material, where the activated length L₂ is less than the initial length L₁. In FIG. 3E, the drawing depicts an example of an artificial chordae tendinae following activation of the shape memory material, where the activated length L₂ is greater than the initial length L₁.

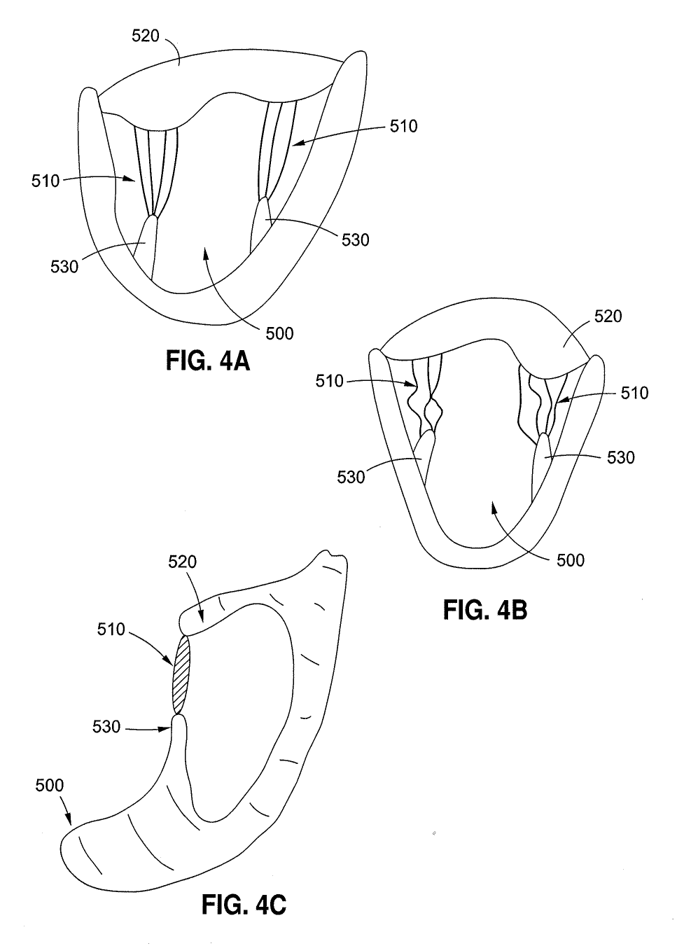

Embodiments of the artificial chordae tendinae as described herein can be configured for use in supporting any of the cardiac valves. In one example, and as illustrated in FIG. 4A, in a left ventricle 500, an artificial chordae tendinae 510 can be attached at one end of a mitral valve leaflet 520, and at an opposite end to a papillary muscle 530. In FIG. 4B, an example is shown of a configuration of the artificial chordae tendinae following activation of the shape memory portion, where the configuration of the left ventricle are altered by the tension exerted upon activation and shortening of the artificial chordae tendinae.

Although not necessarily depicted in the drawing, it will be understood by those skilled in the art that the tension exerted by the artificial chordae tendinae will be effective to improve coaptation of cardiac valve leaflets, and prevent regurgitation as the various chambers of the heart contract and relax during a cardiac cycle. FIG. 4C depicts an alternative view of the left ventricle 500, where the artificial chordae tendinae 510 is attached at one end to a mitral valve leaflet 520, and at the other end to a papillary muscle 530.

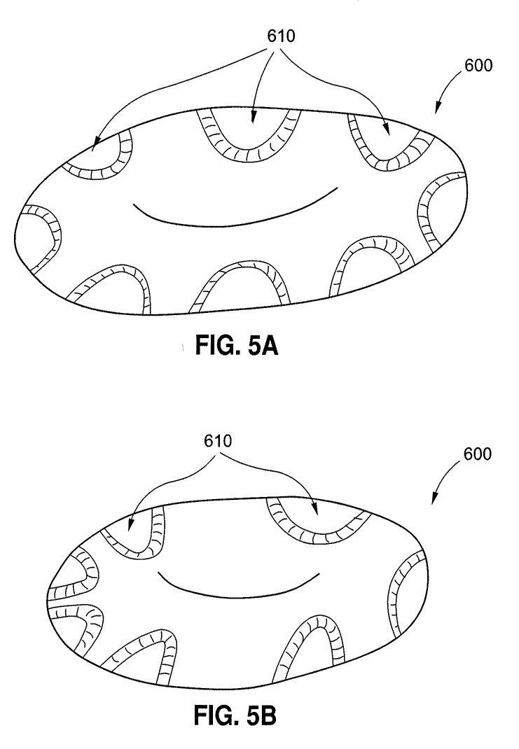

FIG. 5A depicts an example of an adjustable suture 610, configured to be adjusted postoperatively and placed around a mitral valve annulus 600, prior to adjustment. The suture can comprise a shape memory portion that can be activated to change the suture from a first configuration to a second configuration upon application of an energy source to the suture. FIG. 5B depicts and example of an adjustable suture 610 like that described in FIG. 5A, following activation. Here, activation of the shape memory portion of the suture 610 changes its configuration such that the suture exerts a tension on the valve annulus, pulling in the annulus to better support the valve leaflets. In the illustrated embodiment a mitral valve is shown, although the suture 610 is not limited to use with only mitral valves. Any cardiac valve annulus can be effectively repaired using the adjustable suture 610.

In some embodiments, the artificial chordae implant can include an optional cover, or sleeve, that covers all or a portion of the implant. The cover can comprise a biocompatible material, such as silicone; a biodegradable material; and/or a thermal or electrical insulator. In some embodiments, there can be gaps in the covering to permit access to the body of the implant.

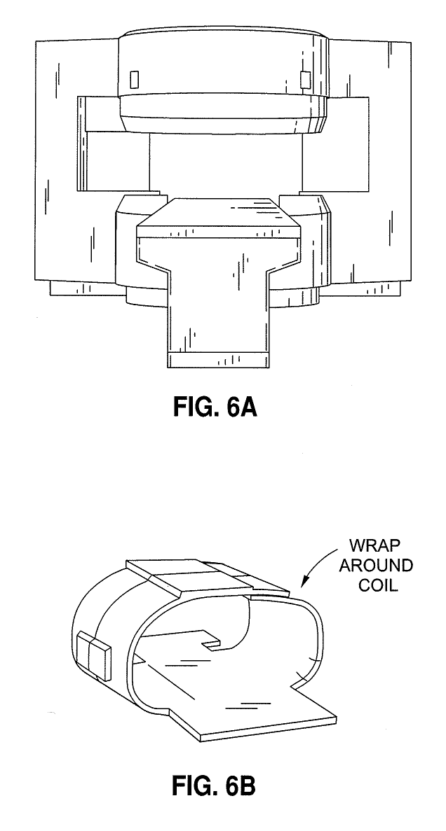

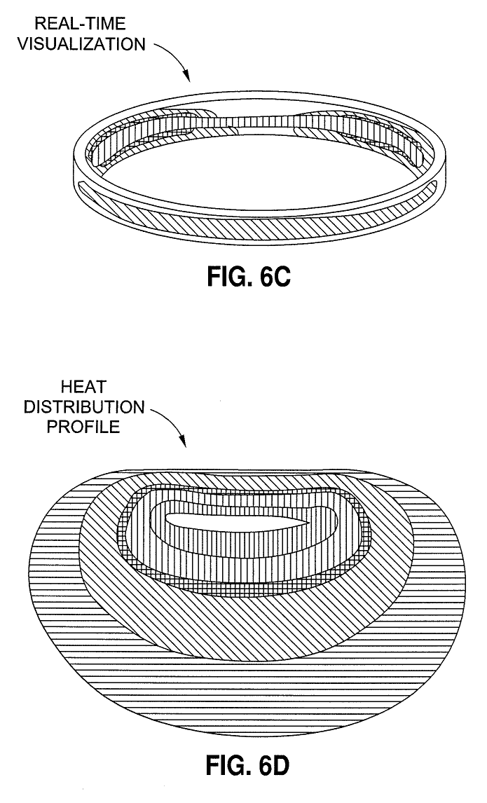

FIGS. 6A-D illustrate an example of activation of a dynamically adjustable artificial chordae tendinae implant via a device configured to focus energy onto the implant. In some embodiments an externally located coil that wraps around the patient can be used to focus magnetic or RF energy onto the implant. FIG. 6C shows a real time image of energy being focused onto an artificial chordae tendinae implant by the coil device of FIGS. 6A and 6B. FIG. 6D shows an image of a heat distribution profile generated in the vicinity of the implant by the wrap around coil.

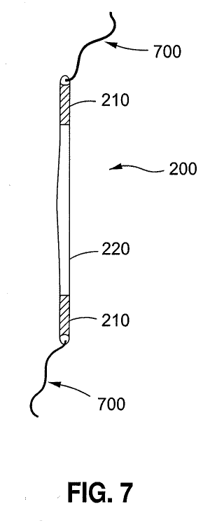

In some embodiments, the implant can further comprise at least one suture, configured for use in securing the implant to a heart valve leaflet and a papillary muscle, or to a valve leaflet and some other anchoring structure in the heart. As shown in FIG. 7, a suture can be provided at each end of the implant 200. In some embodiments, a suture 700 can be provided at locations along the implant other than at the ends. An implant that includes a suture can comprises one or more adjustable portions, for example, shape memory regions 220 that can be adjusted as described above. In some embodiments, an implant that includes a suture can include a cover 210. Those of skill in the art will readily appreciate the various positions where a suture or sutures can be placed along the body of the implant. The embodiments depicted in FIG. 7 is therefore not limiting to the scope of implants that comprises sutures.

In some embodiments, an artificial dynamically adjustable chordae tendinae further comprises an energy absorbing material to increase the rate of heating of the implant while minimizing heating of surrounding tissues adjacent to the implant. Energy absorbing materials for light or laser activation energy can include nanoshells, nanospheres and the like, particularly where infrared laser energy is used to energize the material. These nanoparticles can be made from a dielectric, such as silica, coated with an ultra thin layer of a conductor, such as gold, and be selectively tuned to absorb a particular frequency of electromagnetic radiation. In some embodiments, the nanoparticles range in size between about 5 nm and about 20 nm and can be suspended in a suitable material or solution, such as saline solution. Coatings comprising nanotubes or nanoparticles can also be used to absorb energy from, for example, HIFU, MRI, inductive heating, or the like. The use of an energy absorbing material can be effective to prevent, or at least limit, damage to the surrounding tissues during activation of the implant, by directing more of the applied energy to the implant itself.

In some embodiments, thin film deposition or other coating techniques such as sputtering, reactive sputtering, metal ion implantation, physical vapor deposition, and chemical deposition can be used to cover all or parts of the dynamically adjustable artificial chordae tendinae. Such coatings can be either solid or microporous. When HIFU energy is used, for example, a microporous structure traps and directs the HIFU energy toward the shape memory material. The coating improves thermal conduction and heat removal. In certain embodiments, the coating also enhances radio-opacity of the dynamically adjustable artificial chordae tendinae implant.

Coating materials can be selected from various groups of biocompatible organic or non-organic, metallic or non-metallic materials such as titanium nitride (TiN), iridium oxide (IrO₂), carbon, platinum black, titanium carbide (TiC) and other materials used for pacemaker electrodes or implantable pacemaker leads. Other materials discussed herein or known in the art can also be used to absorb energy.

In addition, in some embodiments, conductive wires such as platinum-coated copper, titanium, tantalum, stainless steel, gold, and the like, can be wrapped around the shape memory material to focus energy and increase the rate of heating of the shape memory material, while reducing heating of surrounding tissues adjacent to the implant.

In some embodiments, the energy source is applied in the course of the surgical procedure, for example after placement of the implant, but before closing the patient. For example, the shape memory material can be heated during implantation of the adjustable artificial chordae tendinae by contacting the artificial chordae tendinae implant with a warm object. In some embodiments, the energy source can be applied after the dynamically adjustable artificial chordae tendinae has been implanted by percutaneously inserting a catheter into the patient's body and applying the energy through the catheter. Any elongated member can be suitably substituted for the exemplary catheter to apply energy through. RF energy, light energy, electrical energy, magnetic energy, thermal energy and the like can be transferred to the shape memory material comprising the adjustable portion of the implant, through a catheter or other elongated object positioned in contact with, or near, the shape memory material.

In some embodiments, thermal energy can be provided to the shape memory material by injecting a heated fluid through a catheter or circulating the heated fluid in a balloon through the catheter placed in close proximity to the shape memory material. In some embodiments, the shape memory material can be coated with a photodynamic absorbing material which is activated to heat the shape memory material when illuminated by light from a laser diode applied directly, or transmitted to the coating through fiber optic elements in a catheter or other like device. In some embodiments, the photodynamic absorbing material includes one or more photoactivatable compounds that are released when illuminated by the laser light. In some embodiments these photoactivatable compounds are effective to promote healing, or to reduce inflammation, following the surgical procedure, in a tissue in the vicinity of the implant.

In some embodiments, a removable subcutaneous electrode or coil couples energy from a dedicated activation unit. In some embodiments, the removable subcutaneous electrode can provide telemetry and power transmission between the system and the dynamically adjustable artificial chordae tendinae. The subcutaneous removable electrode allows more efficient coupling of energy to the implant with minimum or reduced power loss. In certain embodiments, the energy can be delivered via inductive coupling.

In some embodiments, the energy source can be applied in a non-invasive manner from outside the patient's body. In some embodiments, the external energy source can be focused to provide directional heating to the shape memory material so as to reduce or minimize damage to the surrounding tissue. For example, in some embodiments, a handheld or portable device comprising an electrically conductive coil generates an electromagnetic field that non-invasively penetrates the patient's body and induces a current in the dynamically adjustable artificial chordae tendinae. The implant can be configured to include a electrical resistance wire that heats up in response to the induced current flow, resulting in heating of the dynamically adjustable artificial chordae tendinae, and activation of the shape memory material such that it transforms to a memorized shape. In some embodiments, the dynamically adjustable artificial chordae tendinae ring can comprise an electrically conductive coil wrapped around or embedded in the memory shape material. An externally generated electromagnetic field induces a current in the coil of the artificial chordae tendinae, causing it to heat. In some embodiments, where an energy absorbing material is used, the energy absorbing material is configured to transfer thermal energy to the shape memory portion of the implant.

In some embodiments, an external high intensity focused ultrasound (HIFU) transducer focuses ultrasound energy onto the implanted dynamically adjustable artificial chordae tendinae to heat the shape memory material. In some embodiments, the external HIFU transducer can be a handheld or other portable device. The terms “HIFU,” “high intensity focused ultrasound” or “focused ultrasound” as used herein are broad terms and are used at least in their ordinary sense and can include, without limitation, acoustic energy within a wide range of intensities and/or frequencies. For example, HIFU includes acoustic energy focused in a region, or focal zone, having an intensity and/or frequency that is considerably less than what is currently used for ablation in medical procedures. Thus, in some embodiments, the application of focused ultrasound will not result in damage to the patient's cardiac tissue. In some embodiments, HIFU includes acoustic energy within a frequency range of approximately 0.5 MHz and approximately 30 MHz and a power density within a range of approximately 1 W/cm² and approximately 500 W/cm².

In some embodiments, the dynamically adjustable artificial chordae tendinae comprises an ultrasound absorbing material or hydro-gel material that can be configured to heat rapidly when exposed to the ultrasound energy, and to efficiently transfer thermal energy to the shape memory material. In some embodiments, a HIFU probe can be configured to include an adaptive lens system that is able to compensate for heart and/or respiration movements. An adaptive lens system can have multiple focal point adjustments. In some embodiments, a HIFU probe with adaptive capabilities comprises a phased array or linear configuration. In some embodiments, an external HIFU probe comprises a lens configured to be placed between a patient's ribs to improve acoustic window penetration and to address issues and challenges with respect to the passage of acoustic energy through bone.

In some embodiments, HIFU energy is synchronized with an ultrasound imaging device to allow visualization of the dynamically adjustable artificial chordae tendinae implant during HIFU activation. In some embodiments, ultrasound imaging can be used to non-invasively monitor the temperature of tissue surrounding the dynamically adjustable artificial chordae tendinae using velocity of ultrasound, as described in U.S. Pat. No. 4,452,081 (Seppi), the entire contents of which are hereby incorporated herein by reference.

In some embodiments, non-invasive energy can be applied to the artificial chordae implant from a location outside the patient's body. For example, the shape memory material can be activated by a magnetic field generated by an MRI device. In some embodiments, the MRI device generates RF pulses that induce current in a properly configured dynamically adjustable artificial chordae tendinae, resulting in heating of the shape memory material. The dynamically adjustable artificial chordae tendinae can include one or more coils and/or MRI energy absorbing material to increase the efficiency and directionality of the heating. Suitable energy absorbing materials for magnetic activation energy include particulates of ferromagnetic material. Suitable energy absorbing materials for RF energy can include, without limitation, ferrite materials as well as other materials configured to absorb RF energy at particular resonant frequencies. As described above, ultrasound applied from a location outside the patient's body can be used to activate the shape memory portion of the implant.

In some embodiments, an MRI system can be used to measure the size of the implanted dynamically adjustable artificial chordae tendinae before, during and/or after the shape memory material has been activated. In some embodiments, the MRI device generates RF pulses at a first frequency to heat the shape memory material, and at a second frequency to image the implanted artificial chordae tendinae. In some embodiments, the artificial chordae tendinae can include an MRI energy absorbing material that heats in response to magnetic fields. In some embodiments the MRI energy absorbing material can be configured to heat when exposed to a first energy, but not when exposed to a second energy. This allows the use of a single MRI system to both activate the shape memory material (with the first energy) and image the implant (with the second energy).

Other imaging techniques known in the art can also be used to determine the size of the implanted dynamically adjustable artificial chordae tendinae including, for example, ultrasound imaging, computed tomography (CT) scanning, X-ray imaging, and the like. In certain embodiments, imaging modalities can serve the dual purpose of providing the energy needed to activate the shape memory portion of the implant. In some embodiments, the system will be configured such that imaging and adjustment of the implant can be performed concomitantly and in real time.

In some embodiments, imaging and resizing of the dynamically adjustable artificial chordae tendinae can be performed as a separate procedure at some point after the surgery has been completed, for example as part of a postsurgical follow-up. In some embodiments, imaging can be performed after the heart and/or pericardium have been closed, but before closing the patient's chest. This allows the surgeon to check, for example, for leakage and to adjust the implant to reduce regurgitation. For example, energy from the imaging device (or from another source as discussed herein) can be applied to the shape memory material so as to at least partially contract the dynamically adjustable artificial chordae tendinae, increasing tension on the valve leaflets, thus improving coaptation and reducing regurgitation to acceptable levels. In some embodiments adjustments can be made in increments, with evaluation of the improvement in valve function made after each incremental activation of the one or more shape memory portions of the implant. Embodiments where the shape memory material is organized as a plurality of segments are especially well adapted for incremental adjustment.

In some embodiments, the implant can be configured such that application of an energy results in a decrease in tension exerted between the papillary muscle and heart valve leaflet. So, for example, where an implant is placed in a child, the implant can be adjusted as the child grows to continue to provide optimal tension.

Where imaging and adjustment are performed at the same time, it can be advantageous to synchronize adjustment steps with particular physiological parameters, for example with pauses in the patient's breathing, or during quieter portions of the cardiac cycle. For example, where using HIFU to image and/or provide the energy for activation of the shape memory portion, as the heart beats, the artificial chordae tendinae implant can move in and out of the area of focused energy. Thus, to reduce damage to the surrounding tissue, the patient's body can be exposed to the HIFU energy only during portions of the cardiac cycle where it is relatively easy to focus the HIFU energy onto the artificial chordae tendinae implant. For example, in some embodiments, activation and/or imaging can be performed when the heart is relatively at rest, for example during all or a portion of the QT interval of the cardiac cycle. In some cases, it can be advantageous to perform imaging and/or adjustment during a period where the patient is instructed to hold their breath, as is commonly done for some other imaging procedures.

In some embodiments, the energy can be gated to be synchronized with a signal that represents the cardiac cycle, for example an electrocardiogram signal. In some embodiments, the synchronization and gating can be configured to allow delivery of energy to the shape memory materials at specific times during the cardiac cycle to avoid or reduce the likelihood of causing arrhythmia or fibrillation during vulnerable periods. For example, the energy can be gated so as to only expose the patient's heart to the energy during the QT interval of the cardiac cycle, by synchronizing energy output with the appearance of the T-wave while recording of an electrocardiogram.

In some embodiments, application of energy can be synchronized with the acquisition of a focused image of the implant. For example, using edge detection software, the system can be configured to takes a continual series of images and analyze them for the appearance of a best image of the implant. By determining the average time between images, it can be possible to synchronize the application of energy in time with the period of time when the implant is expected to be in an optimal plane of “focus” relative to the “spread pattern” of the energy source being applied, for example, where a HIFU source is used to supply energy with which to result in heating of the implant.

As discussed above, shape memory materials include, for example and without being limiting, polymers, metals, and metal alloys including ferromagnetic alloys. Exemplary shape memory polymers useful in constructing embodiments of the present disclosure are described by Langer, et al. in U.S. Pat. Nos. 6,720,402, 6,388,043, and 6,160,084, all of which are hereby incorporated by reference herein in their entireties.

Shape memory polymers respond to changes in temperature by changing to one or more permanent or memorized shapes. In some embodiments, a shape memory polymer undergoes a shape change when heated to a temperature in a range from about 38° C. to about 60° C. In some other embodiments, a shape memory polymer undergoes a shape change when heated to a temperature in a range from about 40° C. to about 55° C. In some embodiments, the shape memory polymer can be configured to have two-way shape memory properties, such that when the shape memory polymer is heated it transforms to a first memorized shape, and when cooled it transforms to a second memorized shape. The shape memory polymer can be cooled, for example and without limitation, by inserting or circulating a cooled fluid through a catheter.

In some embodiments, heating and cooling can be accomplished through the use of a thermoelectric, e.g., Peltier, device coupled to the implant. As known to those of skill in the art, the thermoelectric effect occurs when a current is passed between two dissimilar metals or semiconductors that are connected to each other at two junctions. When current is passed in one direction the first junction heats while the second junction cools, while when current passes in the opposite direction the situation reverses. Thus coupling a Peltier junction to at least a portion of the shape memory material can be effective to permit rapid heating and cooling of the shape memory material simply by altering the direction of current flow in the device.

In some embodiments, the implant can further comprise temperature sensing devices to provide instant real-time information as to the temperature of the shape memory material, portions of the implant other than the shape memory material, and/or tissue adjacent to the implant site. These temperature sensors can be configured to be coupled with the energy application system such that the application of energy to the implant can be highly regulated, permitting careful control of both the activation temperature, as well as the temperature of the surrounding tissue. Controlling the latter provides the additional advantage of reducing or even preventing inadvertent thermal damage to surrounding tissues near the implant.

In some embodiments, an optional control system can be provided that receives output data from sensors. The control system can be configured or programmed to terminate energy delivery to the implant when either a desired temperature in the implant has been achieved, for example an activation temperature, or if the temperature in surrounding tissue exceeds a predetermined value. The control system can either provide an audible or visible warning to the surgeon to terminate delivery, or the control system can be configured to automatically terminate energy upon satisfying some programmed parameter. In some embodiments, the temperatures at which the system will terminate energy delivery can be programmed by the surgeon or other operator of the system, and can take into account the type of material used in the implant, the degree of shape change required, or the particular sensitivity of surrounding tissues.

A control system can comprise a computer based control system that accepts inputs from sensors or other sources (for example user inputs), and provide outputs effective to configure the implant, or to monitor conditions relative to the implant (e.g., regurgitation). A computerized control system can be programmed with parameters of time, energy, temperature, and any other relevant variables to apply energy to an implant, monitor the change in implant, and to monitor temperature of the implant and tissues in thermal communication with the implant (i.e., tissues in near the implant that might be expected to change temperature in response to an energy applied to the implant). Thus, adjustment of the implant can be performed manually, or automatically, depending on the nature of the control system provided with the energy delivery system.

Shape memory materials implanted in a patient's body can be heated non-invasively, for example, using external light energy sources such as infrared, near-infrared, ultraviolet, microwave and/or visible light sources. In some embodiments, the light energy wavelength can selected such that absorption by the shape memory polymer is optimal while absorption of the surrounding tissue is minimized. Coating and other portions of the implant can be selected to absorb particular wavelengths. By choosing coating materials that absorb wavelengths not readily absorbed by surrounding tissues, the risk of damage by direct interaction of the light energy with tissues adjacent to the implant can be reduced or eliminated.

In some embodiments, the shape memory polymer comprises gas bubbles or bubble-containing liquids such as fluorocarbons that generate bubbles as a result of cavitation of the gas/liquid by HIFU energy.

Certain metal alloys have shape memory qualities and respond to changes in temperature and/or exposure to magnetic fields, or other forms of energy. Exemplary shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

In other embodiments, the shape memory polymer can be heated using electromagnetic fields. As with other forms of energy, specific coatings, layers or other materials can be included in the construction of the implant that improve absorption of electromagnetic energy resulting in an increased rate of heating of the shape memory material, and reduced risk of thermal damage to surrounding tissues.

Shape memory alloys can exist in two distinct solid phases known as martensite and austenite. The martensite phase is relatively soft and easily deformed, whereas the austenite phase is relatively strong and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (Aₛ) and finish transforming to the austenite phase at a finish temperature (A_{f}). Both transformations have a hysteresis. Thus, the Mₛ temperature and the A_{f} temperature are not coincident with each other, and the M_{f} temperature and the Aₛ temperature are not coincident with each other. Thereafter, when the shape memory alloy is exposed to a temperature elevation and transformed to the austenite phase, the alloy changes in shape from the deformed shape to the memorized shape.

Activation temperatures at which the shape memory alloy causes the shape of the artificial chordae tendinae filament to change shape can be selected and built into the implant. Temperatures can be further selected to minimize the amount of energy required to achieve the desired shape change, advantageous in preventing damage to adjacent tissues. Exemplary A_{f} temperatures for suitable shape memory alloys range from about 45° C. to about 70° C. Furthermore, exemplary Mₛ temperatures range from about 10° C. to about 20° C., and exemplary M_{f} temperatures range from about −1° C. to about 15° C. The design of the shape memory material can be such that the implant cannot spontaneously transform from the martensite to austentite configuration without the intervention of the surgeon. The configuration of the artificial chordae can be changed all at once or incrementally in small steps at different times in order to achieve the adjustment necessary to produce the desired clinical result.

In some embodiments, a shape memory alloy can be configured to have a rhombohedral phase, between the austenite and martensite phases, with a rhombohedral start temperature (Rₛ) and a rhombohedral finish temperature (R_{f}). An example of such a shape memory alloy is a NiTi alloy commercially available from Memry Corporation (Bethel, Conn.). In some embodiments, the Rₛ temperature ranges from about 30° C. to about 50° C., and the R_{f} temperature ranges from about 20° C. to about 35° C. An advantage of a material with a rhombohedral phase is that in the rhomobohedral phase the shape memory material can experience a partial physical distortion, compared to the generally rigid structure of the austenite phase, and the generally deformable structure of the martensite phase.

Some shape memory alloys exhibit a ferromagnetic shape memory effect, wherein the shape memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the heart. Furthermore, ferromagnetic materials can include one or more polymer-bonded magnets, wherein magnetic particles are bound within a polymer matrix, such as a biocompatible polymer. The magnetic materials can comprise isotropic and/or anisotropic materials, such as for example neodymium-iron-boron, samarium-cobalt, ferrite and/or aluminum nickel cobalt, also known as rare earth magnetic materials.

In some embodiments, a shape memory material used in an artificial chordae tendinae implant is processed to form a memorized shape that in the austenite phase will substantially replicate the form of a chordae tendinae filament. The shape memory material is then cooled below the M_{f} temperature, where it enters the martensite phase, and is then deformed into a different configuration, for example one suitable for packaging in a percutaneous delivery device. The artificial chordae can be configured such that it provides a measure of repair even before activation. For example, in some embodiments, the shape memory alloy is formed into an artificial chordae tendinae filament that is larger than the memorized shape but still small enough to improve leaflet coaptation and reduce regurgitation. Activation can then be used to further tailor the artificial chordae to the needs of the particular patient.

In some embodiments, the shape memory alloy is sufficiently malleable in the martensite phase to allow a user such as a physician to manually adjust the artificial chordae tendinae filament to a desired length, while still in the martensite phase. After the artificial chordae tendinae filament is placed and coupled between a mitral valve leaflet and papillary muscle, the length or width of the artificial chordae tendinae filament can be adjusted by heating the shape memory alloy to an activation temperature (e.g., temperatures ranging from the Aₛ temperature to the A_{f} temperature). In some embodiments, the surgeon can activate the shape memory material during surgery. In some embodiments, the shape memory material can be activated postoperatively.

In some embodiments, the shape memory material can be transformed from a first configuration to a memorized shape by the application of thermal energy. In some embodiments, an adjustable artificial chordae tendinae comprising a ferromagnetic shape memory alloy can be implanted in a first configuration having a first shape and later changed to a second configuration having a second (e.g., memorized) shape without heating the shape memory material above the Aₛ temperature. Where using ferromagnetic shape memory material an additional advantage is provided in the material can be adjusted more quickly and more uniformly than is typically possible when using shape memory materials that transform to their memorized shape upon heating.

Exemplary ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials can also change shape in response to changes in temperature. Thus, the shape of such materials can be adjusted by exposure to a magnetic field, by changing the temperature of the material, or both. Thus, in some embodiments, a shape memory material can be transformed from an initial conformation to a first memorized shape by the application of one energy, and then to a second memorized shape in response to application of a second energy.

In some embodiments, combinations of different shape memory materials can be used. For example, adjustable artificial chordae tendinae can comprise a combination of shape memory polymer and shape memory alloy (e.g., NiTi, etc.). In some embodiments, the implant can comprise a shape memory polymer tube and a shape memory alloy (e.g., NiTi, etc.) disposed within the tube. Such embodiments are flexible and allow the size and shape of the shape memory portion of the implant to be further reduced without impacting fatigue properties. In addition, or in other embodiments, shape memory polymers are used with shape memory alloys to create a bi-directional (e.g., capable of expanding and contracting) dynamically adjustable artificial chordae tendinae. Bi-directional dynamically adjustable artificial chordae tendinae can be created with a wide variety of shape memory material combinations having different characteristics.

In some embodiments of a method of providing a dynamically adjustable chordae tendinae implant, an imaging module can also be included. Imaging modalities can include, for example, and without limitation, CT, MRI, and ultrasound. Imaging can be used by the surgeon to evaluate the adjustment of the implant prior to completion of the surgery, either by directly viewing the shape of the device, and/or evaluating functional parameters such as improved flow characteristics indicative of a reduction in regurgitation. For example, in some embodiments, Doppler ultrasound can be used to quantify mitral valve regurgitation. Those of skill in the art will readily understand how to perform such measurements (e.g., Dujardin et al. (1997) Circulation 96: 3409-3415).

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein.

Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A dynamically adjustable artificial chordae tendinae implant, for use in treating a heart valve in a patient, comprising:
a body portion, having first and second ends, and comprising:
a first attachment portion that couples the body portion to a leaflet of a valve in a heart;
a second attachment portion that couples the body portion to a papillary muscle in the heart; and
an adjustable portion, comprising a shape memory material;
wherein, in response to an activation energy, the adjustable portion transforms from a first conformation to a second conformation;
wherein in the first conformation the ends of body portion are separated by a first length; and
wherein in the second conformation the ends of the body portion are separated by a second length.

2. The implant of claim 1, wherein transformation from the first conformation to the second conformation results in improved coaptation of the leaflet of the valve with at least one other leaflet of the same valve.

3. The implant of claim 1, wherein the shape memory material comprises at least one of a shape memory alloy, a ferromagnetic shape memory alloy, a shape memory polymer, and a combination thereof.

4. The implant of claim 1, wherein the adjustable portion is configured to transform from the first conformation to the second conformation at a first activation temperature.

5. The implant of claim 4, wherein the adjustable portion is configured to transform to a third conformation at a second activation temperature.

6. The implant of claim 5, wherein, in the third conformation, the ends of the body portion are separated by a third length.

7. The implant of claim 1, wherein the first length is greater than the second length, for at least a portion of a cardiac cycle.

8. The implant of claim 1, wherein the first length is less than the second length, for at least a portion of a cardiac cycle.

9. The implant of claim 6, wherein the third length is greater than the second length, for at least a portion of a cardiac cycle.

10. The implant of claim 6, wherein the third length is greater than the first length, for at least a portion of a cardiac cycle.

11. The implant of claim 6, wherein the third length is less than the first length, for at least a portion of a cardiac cycle.

12. The implant of claim 1, wherein transformation from the first conformation to the second conformation occurs incrementally.

13. The implant of claim 6, wherein transformation to the third conformation occurs incrementally.

14. The implant of claim 1, wherein at least one of the first attachment portion and the second attachment portion comprises a suture.

15. The implant of claim 14, wherein the suture comprises at least one of catgut, silk, linen, stainless steel wire, polyglycolic acid, polyglactin, polydioxanone, polyglyconate, polyamide, polyester, polypropylene, ePTFE, and a combination thereof.

16. The implant of claim 1, further comprising a cover over at least a portion of the implant.

17. The implant of claim 16, wherein the cover comprises at least one of a biodegradable material, a biocompatible material, a thermal insulator, an electrical insulator, and a combination thereof.

18. The implant of claim 17, wherein the cover comprises a gap configured to expose a portion of the implant.

19. The implant of claim 16, wherein the cover can be configured to be suturable to at least one of the valve leaflet and the papillary muscle.

20. The implant of claim 1, further comprising at least one medicament in or on at least a portion of the implant, the medicament effective to promote healing, reduce inflammation, or reduce thrombosis, in the patient.

21. The implant of claim 1, further comprising an energy absorbing material coupled to the adjustable portion.

22. The implant of claim 21, wherein the energy absorbing material is configured to provide thermal energy to the adjustable portion.

23. The implant of claim 21, wherein the energy absorbing material comprises at least one of a hydrogel, carbon, graphite, a ceramic material, a magnetic material, a microporous coating, a magnetic induction coil, an electrically conductive wire, nanospheres, and combinations thereof.

24. The implant of claim 21, wherein the energy absorbing material is configured to absorb at least one of electromagnetic energy, radiofrequency energy, acoustic energy, light energy, thermal energy, electrical energy, mechanical energy, and a combination thereof.

25. The implant of claim 24, wherein the acoustic energy comprises high intensity focused ultrasound energy.

26. The implant of claim 1, wherein the implant comprises a plurality of adjustable portions, each of the plurality of adjustable portions comprising a shape memory material;
wherein, in response to an activation energy, each of the plurality of adjustable portions transforms from an initial conformation to a transformed conformation.

27. The implant of claim 26, wherein each of the plurality of adjustable portions transforms independently from the initial conformation to the transformed conformation.

28. The implant of claim 26, wherein the plurality of adjustable portions are arranged in segments along at least a portion of the body portion.

29. The implant of claim 28, wherein each adjustable portion segment is separated from an adjacent adjustable portion segment by a non-adjustable portion.

30. The implant of claim 29, wherein the non-adjustable portion comprises an insulator.

31. The implant of claim 1, further comprising at least one sensor configured to output data to a receiver, the data indicative of at least one of a temperature of the implant and a temperature of a body tissue in thermal communication with the implant.

32. A dynamically adjustable artificial chordae tendinae implant system, comprising:
an implant, comprising:
a body portion, having first and second ends, and comprising:
a first attachment portion that couples the body portion to a leaflet of a valve in a heart;
a second attachment portion that couples the body portion to a papillary muscle in the heart; and
an adjustable portion, comprising a shape memory material;
wherein, in response to an activation energy, the adjustable portion transforms from a first conformation to a second conformation;
wherein in the first conformation the ends of body portion are separated by a first length; and
wherein in the second conformation the ends of the body portion are separated by a second length; and
a energy delivery system configured to deliver the activation energy to the implant.

33. The system of claim 32, wherein the energy delivery system delivers at least one of electromagnetic energy, radiofrequency energy, acoustic energy, light energy, thermal energy, electrical energy, and mechanical energy to the implant.

34. The system of claim 32, further comprising at least one sensor configured to output data indicative of at least one of a temperature of the implant and a temperature in a tissue in thermal communication with the implant.

35. The system of claim 34, wherein the energy delivery system is configured to terminate or reduce energy delivery upon receipt of data from the at least one sensor indicative of at least one of attaining a target temperature in the implant and exceeding a threshold temperature in the tissue.

36. The system of claim 34, further comprising a display module for displaying the data.

37. A dynamically adjustable artificial chordae tendinae implant system, comprising:
coupling means for coupling a heart valve leaflet to a papillary muscle in a patient, the coupling means having first and second ends separated by a first length;
adjusting means for changing the length of the coupling means;
wherein the adjusting means comprises a shape memory material that transforms from a first conformation to a second conformation in response to an activation energy; and
wherein, when the shape memory material transforms from the first conformation to the second conformation, the implant improves coaptation of the heart valve leaflet with at least one other heart valve leaflet.

38. The system of claim 37, configured such that when the shape memory material is in the second conformation, the ends of the coupling means are separated by a second length.

39. The system of claim 37, further comprising energy delivery means for delivering the activation energy to the implant.

40. The system of claim 39, wherein the activation energy is at least one of electromagnetic energy, radiofrequency energy, acoustic energy, light energy, thermal energy, electrical energy, mechanical energy, and a combination thereof.

41. The system of claim 39, wherein the energy delivery means is configured to deliver the activation energy of the implant from a location outside the patient's body.

42. The system of claim 37, further comprising sensing means for outputting data indicative of at least one of a temperature of the implant and a temperature of a tissue in thermal communication with the implant.

43. The system of claim 42, further comprising display means for displaying the at least one of the temperature of the implant and the temperature of the tissue.

44. The system of claim 42, further comprising control means for terminating or reducing delivery of the energy to the implant in response to output data from the sensing means indicative of at least one of achieving a target temperature in the implant and exceeding a threshold temperature in the tissue.

45. The system of claim 37, wherein the shape memory material comprises at least one of a shape memory alloy, a ferromagnetic shape memory alloy, a shape memory polymer, and a combination thereof.

46. The system of claim 37, wherein the shape memory material is configured to transform from the first conformation to the second conformation at a first activation temperature.

47. The system of claim 38, wherein the shape memory material is configured to transform to a third conformation at a second activation temperature.

48. The system of claim 47, wherein, when the shape memory material is in the third conformation, the ends of the coupling means are separated by a third length.

49. The system of claim 47, further comprising attachment means for attaching the implant to at least one of the heart valve leaflet and the papillary muscle.

50. The system of claim 37, wherein the attachment means comprises a suture.

51. The system of claim 37, further comprising a covering means for covering at least a portion of the coupling means.

52. The system of claim 51, wherein the covering means comprises at least one a biodegradable material, a biocompatible material, and an insulator.

53. The system of claim 37, wherein the implant comprises a plurality of adjusting means, each of the plurality of adjusting means comprising a shape memory material;
wherein, in response to an activation energy, each of the plurality of adjusting means transforms from an initial conformation to a transformed conformation.

54. The system of claim 53, wherein each of the plurality of adjusting means transforms independently from the initial conformation to the transformed conformation.

55. The system of claim 53, wherein the plurality of adjusting means are arranged in segments along at least a portion of the coupling means.

56. The system of claim 53, further comprising separating means for separating each of the plurality of adjusting means.

57. The system of claim 56, wherein the separating means comprises a thermal insulator.

58. A method, for implanting an artificial chordae tendinae in a patient, comprising:
providing a dynamically adjustable artificial chordae tendinae implant, comprising:
a body portion, having first and second ends, and comprising:
a first attachment portion that couples the body portion to a leaflet of a valve in a heart;
a second attachment portion that couples the body portion to a papillary muscle in the heart; and
an adjustable portion, comprising a shape memory material;
wherein, in response to an activation energy, the adjustable portion transforms from a first conformation to a second conformation;
wherein in the first conformation the ends of body portion are separated by a first length; and
wherein in the second conformation the ends of the body portion are separated by a second length;
securing the first attachment portion to the heart valve leaflet;
securing the second attachment portion to the papillary muscle;
delivering the activation energy to the adjustable portion of the implant, resulting in a transformation from the first conformation to the second conformation;
wherein transformation from the first conformation to the second conformation results in improved coaptation of the leaflet of the cardiac valve with at least one other leaflet of the same cardiac valve.

59. The method of claim 58, wherein the activation energy comprises at least one of electromagnetic energy, radiofrequency energy, acoustic energy, light energy, thermal energy, electrical energy, mechanical energy, and a combination thereof.

60. The method of claim 59, wherein the activation energy is delivered from outside the patient's body.

61. The method of claim 58, further comprising providing at least one sensor configured to output data corresponding to at least one of a temperature of the implant and a temperature of a tissue in thermal communication with the implant.

62. The method of claim 61, further comprising terminating or reducing the delivery of activation energy to the implant in response to output data from the at least one sensor indicative of at least one of achieving a target temperature in the implant and exceeding a threshold temperature in the tissue.
